(19) United States
(12) Patent Application Publication
(10) Pub. No.: US 2013/0197625 A1
(43) Pub. Date: Aug. 1, 2013

(54) FIBERS AND YARNS USEFUL FOR CONSTRUCTING GRAFT MATERIALS

(71) Applicant: Cordis Corporation, Bridgewater, NJ (US)

(72) Inventor: Clifford J. DWYER, Weston, FL (US)

(73) Assignee: Cordis Corporation, Bridgewater, NJ (US)

(21) Appl. No.: 13/795,156

(22) Filed: Mar. 12, 2013

Related U.S. Application Data
(62) Division of application No. 11/356,677, filed on Feb. 17, 2006.

(51) Int. Cl. B32B 7/02; A61F 2/06

(52) U.S. Cl. CPC B32B 7/02; A61F 2/06
USPC 623/1.32; 428/212; 600/36

(57) ABSTRACT

The present invention discloses a composite yarn comprising at least one wear-resistant polymeric fiber and at least one flexible polymeric fiber. The present invention also discloses a co-extruded filament comprising a polymeric inner core and a polymeric outer sheath. The polymeric inner core comprises a flexible polymeric material and the polymeric outer sheath comprises a wear-resistant polymeric material. The composite yarn and the co-extruded filament synergistically combine durability and flexibility, and thereby are particularly useful for the construction of graft materials. The present invention further discloses a reinforced fiber graft comprising wear-resistant beads and weaves of flexible polymeric fibers. In another aspect, the present invention discloses a process for assembling a graft device without suture knots by using the inventive co-extruded filament.

### FIELD OF INVENTION

The present invention relates to fibers and yarns useful as graft materials in vascular grafts or other graft devices. Particularly, the present invention relates to a composite yarn, a co-extruded filament, and a reinforced fiber graft material. The present invention also relates to a process for assembling a graft device without using suture knots.

### BACKGROUND OF INVENTION

Graft devices have been widely used to replace malfunctioning biological structures or treat diseases associated therewith. For example, various vascular grafts are now Food and Drug Administration (FDA) approved and commercially available for treating a wide range of vascular diseases. A vascular graft typically comprises one or more stent segments, a graft material, and suture knots which are tied in such a way to affix the graft material to an outside portion of the one or more stent segments. The stent segment is either an expandable wire mesh or hollow perforated tube. The graft material is formed by fibers or yarns of biocompatible materials through a weaving, knitting, or braiding process.

One major challenge for developing a graft device, particularly, a vascular graft, is the lack of appropriate graft materials. The biostability and biocompatibility of the graft materials are critical for the use of graft devices. Since vascular grafts are intended for prolonged or permanent use and directly interface with body tissue, body fluids, and various biological molecules, the graft materials thereof must meet stringent biological and physical requirements.

When placed within the body in vessels, due to pulsatile blood pressure, a vascular graft, particularly, the graft material, is subject to high hydrodynamic forces and relative motion or rubbing between the stent segments and the graft material. Essentially, these forces and relative motion tend to wear the graft material at the points where it is connected to the stent segments. Over time, the graft material may develop microleaks which significantly undermine the performance of the vascular graft. Therefore, the graft material is required to be wear-resistant and highly durable. Furthermore, a vascular graft needs to conform to the anatomy of the patient's body without inducing detrimental stress. Thus, the graft material is required to be flexible and lubricious.

To achieve desirable flexibility and durability, prior art graft materials utilize yarns possessing different properties in blend. For example, a yarn of a flexible material and a yarn of a wear-resistant material may be used in combination to form a graft material. However, to satisfy the desired flexibility and durability, graft materials formed by blending different yarns are often too bulky. Since vascular grafts are mainly utilized to establish a fluid flow path from one section of a blood vessel to another section of the same or different blood vessel, it is preferred that a vascular graft has a low profile, i.e., small size. Lower profile also improves the maneuverability of a vascular graft. The size of a vascular graft can be reduced by employing thin-walled graft materials and/or decreasing the number of suture knots. However, the durability of thin-walled graft materials formed by conventional fibers or yarns is substantially less than that of graft materials having standard thickness. Furthermore, suture knots are important for securing the connection and minimizing the relative motion between the stent segments and the graft material. In a conventional vascular graft, almost each strut of each stent segment is secured to the graft material by suture knots.

Therefore, there remains a need for a fiber or yarn that can form flexible, wear-resistant, highly durable, and thin-walled graft materials.

### SUMMARY OF THE INVENTION

Accordingly, the present invention provides a composite yarn for construction of graft materials comprising at least one wear-resistant polymeric fiber and at least one flexible polymeric fiber. In the inventive composite yarn, the total number of the at least one wear-resistant polymeric fiber and the at least one flexible polymeric fiber ranges from about 5 to about 150, and the ratio of the at least one wear-resistant polymeric fiber to the at least one flexible polymeric fiber by number is about 1:4 to about 4:1. Preferably, the total number of the at least one wear-resistant polymeric fiber and the at least one flexible polymeric fiber ranges from about 10 to about 50.

The present invention also provides a co-extruded filament comprising a polymeric inner core and a polymeric outer sheath. The polymeric inner core comprises a flexible polymeric material and the polymeric outer sheath comprises a wear-resistant polymeric material. The melting point of the polymeric outer sheath is lower than the melting point of the polymeric inner core.

In another aspect, the present invention provides a process for assembling a graft device. The inventive process comprises steps of: providing one or more scaffold structures; providing a graft material, which is formed by a co-extruded filament comprising a polymeric inner core and a polymeric outer sheath, wherein the polymeric inner core comprises a flexible polymeric material and the polymeric outer sheath comprises a wear-resistant polymeric material, and the melting point of the polymeric outer sheath is lower than the melting point of the polymeric inner core; placing the graft material in contact with an outside portion of the one or more scaffold structures to form a scaffold-graft assembly; and heating the scaffold-graft assembly to affix the graft material to the outside portion of the one or more scaffold structures.

The present invention also provides a reinforced fiber graft material comprising wear-resistant beads and weaves of flexible polymeric fibers, wherein the wear-resistant beads are attached to the flexible polymeric fibers.

### BRIEF DESCRIPTION OF THE DRAWINGS

FIGS. 1A to 1D are pictorial cross-section illustrations of four embodiments of the inventive composite yarn.

FIG. 2 is a pictorial cross-section illustration of one embodiment of the inventive co-extruded filament.

FIG. 3A is a pictorial illustration of a conventional abdominal aortic aneurysm (AAA) device having suture knots; and FIG. 3B is a pictorial illustration of an AAA device without suture knots.

FIG. 4 is a pictorial illustration of a portion of one embodiment of the inventive reinforced fiber graft material.

### DETAILED DESCRIPTION OF THE INVENTION

The present invention provides a composite yarn for construction of graft materials. The composite yarn comprises at least two types of fibers, i.e., at least one wear-resistant polymeric fiber and at least one flexible polymeric fiber. The total number of the at least one wear-resistant polymeric fiber and the at least one flexible polymeric fiber ranges from about 5 to about 150. Preferably, the total number of the at least one wear-resistant polymeric fiber and the at least one flexible polymeric fiber ranges from about 10 to about 50. The ratio of the at least one wear-resistant polymeric fiber to the at least one flexible polymeric fiber by number is about 1:4 to about 4:1. The term “yarn” as used herein denotes a long continuous length of interlocked fibers. The polymeric fiber of the present invention may be a monofilament or a co-extruded filament. By “monofilament”, it is meant a single strand of fiber consisting of one polymeric material. By “co-extruded filament”, it is meant a single strand of fiber formed by extruding at least two polymeric materials together. Preferably, the inventive composite yarn has a denier ranging from about 15 to about 500, and a tensile strength ranging from about 1.5 to about 3.5 Gpa. The term “denier” as used herein is defined as the mass in grams per 9000 meters.

The at least one wear-resistant polymeric fiber may be a fiber of any polymer that is stiff and wear-resistant. By “wear-resistant”, it is meant being capable of withstanding the force or the effect of wear, including adhesive wear, abrasive wear, corrosive wear, and surface fatigue. Polymers suitable for the wear-resistant polymeric fiber of the present invention include, but are not limited to: polyolefin, polyester, poly(ether amide), poly(ether ester), poly(ether urethane), poly(ester urethane), poly(ethylene-styrene/butylene-styrene), and other block copolymers. Preferably, the wear-resistant polymeric fiber is a fiber of ultra high molecular weight polyethylene and ultra high molecular weight polypropylene. As used herein, the terms “ultra high molecular weight polyethylene” and “ultra high molecular weight polypropylene” denote a polyethylene having between six and twelve million ethylene units per molecule and a polypropylene having between six and twelve million propylene units per molecule, respectively. Ultra high molecular weight polyethylene is also known as UHMWPE or Dyneema®.

The at least one flexible polymeric fiber may be a fiber of any polymer that is flexible and lubricous. Polymers suitable for the flexible polymeric fiber of the present invention include, but are not limited to: polyamide, polyester, polyolefin, and fluorinated polymer. Examples of the polymers suitable for the flexible polymeric fiber include, but are not limited to: nylon 6, nylon 66, nylon 11, nylon 12, polyethylene terphthalate, polybutylene terephthalate, low density polypropylene, low density polyethylene, and poly(vinylidene fluoride). Preferably, the flexible polymeric fiber is a fiber of polyethylene terphthalate, or polybutylene terephthalate. Polyethylene terphthalate is also known as Dacron®. The terms “low density polypropylene” and “low density polyethylene” as used herein denote polypropylene and polyethylene, respectively, which have a high degree of short and long chain branching and a density range of about 0.91 to about 0.94 g/cc.

In the present invention, the at least one flexible polymeric fiber imparts flexibility and lubricity to the inventive composite yarn, while the at least one wear-resistant polymeric fiber imparts strength and durability to the inventive composite yarn. Depending on the intended use or performance requirement, the properties of the inventive composite yarn, such as flexibility and durability, may be controlled by fiber material selection and/or fiber strand arrangement. That is, the properties of the inventive composite yarn may be tuned by using different wear-resistant polymeric fiber and flexible polymeric fiber, varying the ratio of the wear-resistant polymeric fiber to the flexible polymeric fiber, and/or adjusting the orientation of the wear-resistant polymeric fiber and the flexible polymeric fiber within the inventive composite yarn. In other words, various amounts of the at least one wear-resistant polymeric fiber and the at least one flexible polymeric fiber may be mixed and positioned to achieve a balance of flexibility and durability for the inventive composite yarn. For example, the flexibility of the inventive yarn can be enhanced by reducing the ratio of the at least one wear-resistant polymeric fiber and the at least one flexible polymeric fiber; while the strength and durability of the inventive yarn can be enhanced by increasing the ratio of the at least one wear-resistant polymeric fiber and the at least one flexible polymeric fiber. When the at least one wear-resistant polymeric fiber is positioned to form a central core, and the at least one flexible polymeric fiber is positioned to form a periphery surrounding the central core, the resulting composite yarn possesses a flexible and lubricous surface and a strong core. Alternatively, when the at least one flexible polymeric fiber is positioned to form a central core, and the at least one wear-resistant polymeric fiber is positioned to form a periphery surrounding the central core, the resulting composite yarn possesses a strong and wear-resistant surface and a flexible core. When the flexibility and durability of the inventive composite yarn need to be evenly balanced, the at least one wear-resistant polymeric fiber and the at least one flexible polymeric fiber can be evenly blended in pairs. In addition, the polymeric fibers in the inventive composite yarn may be co-extruded filaments combining individual properties of various polymeric materials.

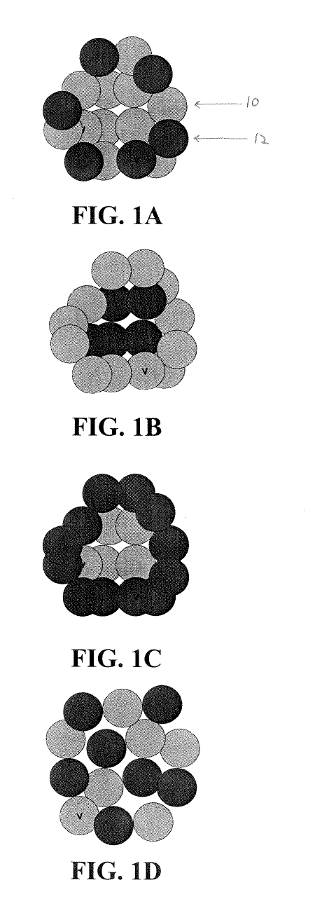

FIGS. 1A to 1D show the cross-section views of four embodiments of the inventive composite yarn that comprise multiple wear-resistant polymeric fibers and multiple flexible polymeric fibers. In FIG. 1A, the inventive composite yarn comprises 6 wear-resistant polymeric fibers and 11 flexible polymeric fibers in a bundle with the wear-resistant polymeric fibers positioned at the periphery of the bundle. In FIG. 1B, the inventive composite yarn comprises 12 wear-resistant polymeric fibers and 5 flexible polymeric fibers in a bundle wherein the flexible polymeric fibers are positioned to form a central core and the wear-resistant polymeric fibers are positioned to form a periphery surrounding the central core. In these drawings, light gray color, i.e., reference number 10, denotes a flexible polymeric fiber, and dark gray color, i.e., reference number 12, denotes a wear-resistant polymeric fiber. In FIG. 1C, the inventive composite yarn comprises 5 wear-resistant polymeric fibers and 12 flexible polymeric fibers in a bundle wherein the wear-resistant polymeric fibers are positioned to form a central core and the flexible polymeric fibers are positioned to form a periphery surrounding the central core. In FIG. 1D, the inventive composite yarn comprises 7 wear-resistant polymeric fibers and 7 flexible polymeric fibers in a bundle wherein the wear-resistant polymeric fibers and the flexible polymeric fibers are blended in pairs. That is, in FIG. 1D, the wear-resistant polymeric fibers and the flexible polymeric fibers are arranged in an even manner to form the inventive composite yarn.

The inventive composite yarn synergistically combines flexibility and durability, and thereby is particularly suitable to be used for the construction of graft materials. Unlike the prior art methods which blend yarns of different materials, the present invention blends various polymeric fibers to form a composite yarn. Comparing to a fabric prepared by blending different yarns to obtain specific characteristics, a fabric prepared from the inventive composite yarn can achieve the same or improved characteristics with reduced fabric thickness. Therefore, when used to construct graft materials for vascular grafts or other graft devices, the inventive composite yarn not only shows improved balance of desired properties, but also minimizes the overall material thickness needed to achieve the desired mechanical properties, thereby providing a thin-walled graft material with enhanced durability. Moreover, the inventive composite yarn provides a more homogeneous blend for graft materials, thus enhancing the performance of the graft material.

In one preferred embodiment of the present invention, the inventive composite yarn comprises polymeric fibers of polyethylene terphthalate and polymeric fibers of ultra high molecular weight polyethylene, wherein the total number of polymeric fibers are 20, and the ratio of polymeric fibers of polyethylene terphthalate and polymeric fibers of ultra high molecular weight polyethylene by number is about 1:4 to about 4:1.

The inventive composite yarn may be prepared through a spinning process, an air texturizing process, or other processes known to one skilled in the art. Methods of preparing composite yarn are well known in the art and detailed conditions for preparing the inventive composite yarn can be readily ascertained by one skilled in the art.

The inventive composite yarn may form graft materials utilizing any number of techniques, including weaving, knitting and braiding. Weaving involves the interlacing, at right angles, of two systems of threads known as warp and filling. Warp threads run lengthwise in a woven fabric and filling threads run cross-wise. Knitting is the process of making fabric by interlocking a series of loops of one or more threads. Braiding involves crossing diagonally and lengthwise several threads of any of the major textile fibers to obtain a certain width effect, pattern or style.

The present invention also provides a co-extruded filament comprising a polymeric inner core and a polymeric outer sheath. The polymeric inner core comprises a flexible polymeric material. The polymeric outer sheath comprises a wear-resistant polymeric material. The melting point of the polymeric outer sheath is lower than the melting point of the polymeric inner core.

Preferably, the inventive co-extruded filament has a denier ranging from about 20 to about 1000 with about 20 to about 300 filaments per bundle. It is also preferred that the inventive co-extruded filament has a tensile strength ranging from about 1.5 to about 3.5 GPa.

The wear-resistant polymeric material of the polymeric outer sheath may be any polymer that is stiff and wear-resistant. Polymers suitable for the wear-resistant polymeric material of the inventive co-extruded filament include, but are not limited to: polyolefin, polyester, poly(ether amide), poly(ether ester), poly(ether urethane), poly(ester urethane), poly(ethylene-styrene/butylene-styrene) and other block copolymers. The terms “ultra high molecular weight polyethylene” and “ultra high molecular weight polypropylene” are the same as defined hereinabove. Preferably, the wear-resistant polymeric material is ultra high molecular weight polyethylene or ultra high molecular weight polypropylene.

The polymeric outer sheath may also further comprise a biodegradable polymer. By “biodegradable polymer”, it is meant a polymer that can be degraded or decomposed by a biological process, as by the action of bacterial, plant, or animal. Examples of biodegradable polymers suitable for the present invention include, but are not limited to: polyvinyl pyrrolidone, polyethylene glycol, polyethylene oxide, polyvinyl alcohol, polyglycol lactic acid, polylactic acid, polycaprolactone, polydioxanone, polyamino acid, and derivatives and mixtures thereof. Biodegradable polymer is also known as “bioabsorbable polymer” or “biodissolvable polymer”. Preferably, the biodegradable polymer is polycaprolactone or polydioxanone.

Optionally, the polymeric outer sheath may further comprise one or more biologically active molecules. The one or more biologically active molecules may be physically impregnated or disperse in or covalently attached to the polymeric outer sheath. The term “biologically active molecule” as used herein denotes a compound or substance having an effect on or eliciting a response from living tissue. The biologically active molecules suitable for the present invention include, for example, any drugs, agents, compounds and/or combination thereof that have therapeutic effects for treating or preventing a disease or a biological organism's reaction to the introduction of the medical device to the organism. Preferred biological active molecules include, but are not limited to: anti-thrombogenic agents, immuno-suppressants, anti-neoplastic agents, anti-inflammatory agents, angiogenesis inhibitors, protein kinase inhibitors, and other agents which may cure, reduce, or prevent restenosis in a mammal. Examples of the biological active molecules of the present invention include, but are not limited to: heparin, albumin, streptokinase, tissue plasminogin activator (TPA), urokinase, rapamycin, paclitaxel, pimecrolimus, and their analogs and derivatives.

The flexible polymeric material of the polymeric inner core may be any polymer that is flexible and lubricous. Polymers suitable for the flexible polymeric material of the inventive co-extruded filament include, but are not limited to: polyamide, polyester, polyolefin, and fluorinated polymer. Examples of the polymers suitable for the flexible polymeric material include, but are not limited to: nylon 6, nylon 66, nylon 11, nylon 12, polyethylene terphthalate, polybutylene terephthalate, low density polypropylene, low density polyethylene, and poly(vinylidene fluoride). The terms “low density polypropylene” and “low density polyethylene” are the same as defined hereinabove. Preferably, the flexible polymeric fiber is a fiber of polyethylene terphthalate, or polybutylene terephthalate.

In the present invention, the polymeric inner core imparts flexibility and lubricity to the inventive co-extruded filament, while the polymeric outer sheath imparts strength and durability to the inventive co-extruded filament. When used in the construction of graft materials, the inventive co-extruded filament not only shows improved balance of desirable properties, but also minimizes the overall material thickness, thereby providing a thin-walled graft material with enhanced durability. Depending on the intended use or performance requirement, the properties of the inventive co-extruded filament, such as flexibility and durability, may be controlled by components material selection and/or ratio of the polymeric inner core to the polymeric outer sheath. That is, the properties of the inventive co-extruded filament may be tuned by using different wear-resistant polymeric material and flexible polymeric material as components, and/or varying the ratio of the polymeric inner core to the polymeric outer sheath. It is preferred that the ratio of the polymeric inner core to the polymeric outer sheath by weight ranges from about 90:10 to about 10:90, with the ratio of about 80:20 more preferred. The inventive co-extruded filament synergistically combines flexibility and durability, and thereby is particularly suitable to be used in vascular grafts or other graft devices.

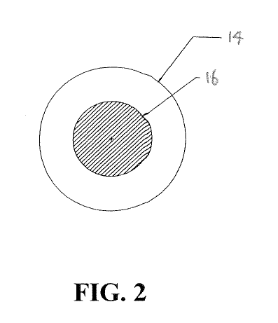

FIG. 2 shows the cross-sectional view of the inventive co-extruded filament that comprises a polymeric inner core and a polymeric outer sheath. In FIG. 2, reference number 14 denotes the outer sheath of a wear-resistant polymeric material, and reference number 16 denotes the inner core of a flexible polymeric material.

In one preferred embodiment of the present invention, the inventive co-extruded filament consists of a polymeric inner core of polyethylene terphthalate and a polymeric outer sheath of ultra high molecular weight polyethylene in a ratio of 80:20 by weight.

Methods of preparing co-extruded filaments are well known in the art and the inventive co-extruded filament may be prepared through suitable processes readily ascertainable by one skilled in the art.

In another aspect, the present invention provides a process for assembling a graft device without using suture knots. The inventive process utilizes a graft material formed by the inventive co-extruded filament. To assemble a graft device, one or more scaffold structures and a graft material formed the inventive co-extruded filaments may be provided in any sequence. The inventive co-extruded filament may form a graft material utilizing any number of techniques as described hereinabove, such as weaving, knitting, and braiding.

The graft material formed by the inventive co-extruded filaments is then placed in contact with an outside portion of the one or more scaffold structures forming a scaffold-graft assembly. A scaffold-graft assembly may also be formed by directly braiding the inventive co-extruded filament on one or more scaffold structures in such a way that the inventive co-extruded filament forms a graft material encapsulating an outside portion of the one or more scaffold structures.

Next, the scaffold-graft assembly is heated to a temperature at which the polymeric outer sheath of the inventive co-extruded filament fuses and thereby attaches to the outside portion of the one or more scaffold structures. That is, the graft material comprising the inventive co-extruded filament is affixed to the outside portion of the one or more scaffold structures through heat. It is preferred that the temperature to which the scaffold-graft assembly is heated is about the melting point of the polymeric outer sheath or above but lower than the melting point of the polymeric inner core. In one embodiment of the present invention, the temperature to which the scaffold-graft assembly is heated is about 155° C. or below.

Comparing to graft devices assembled by affixing graft materials to scaffold structures through tying knots with sutures, graft devices assembled by the inventive process have a lower profile. In addition, the inventive process reduces the complexity of assembling graft devices and can be automated, thereby significantly improving efficiency and productivity of graft device assembly processes.

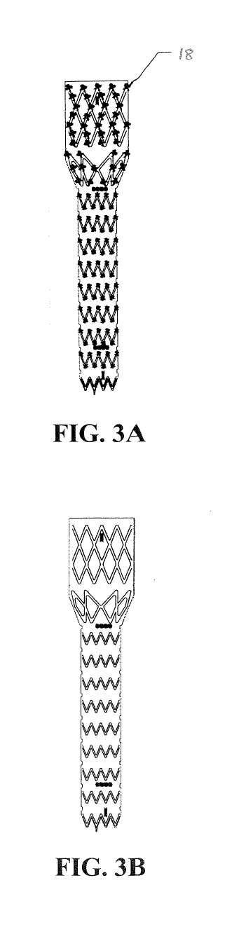

FIG. 3A is a pictorial illustration of a conventional abdominal aortic aneurysm (AAA) device comprising a stent, a graft material, and suture knots. In the conventional AAA device, the graft material is affixed to the stent through suture knots. In FIG. 3A, reference number 18 denotes a suture knot. FIG. 3B is a pictorial illustration of an AAA device assembled by the inventive process. The AAA device in FIG. 3B does not contain any suture knot.

In one embodiment of the present invention, a scaffold-graft assembly is formed by contacting a graft material to an outside portion of one or more scaffold structures; then the scaffold-graft assembly is placed inside an induction-heating coil to raise the temperature of the one or more scaffold structures without heating the whole graft material. This induction-heating process allows preservation of the original porosity of the graft material.

In another embodiment of the present invention, the inventive co-extruded filament is directly braided on one or more scaffold structures in such a way that the inventive co-extruded filament forms a graft material encapsulating the one or more scaffold structures; then the graft material is heated to fuse the polymeric outer sheath of the inventive co-extruded filament thus attaching the graft material to the outside portion of the one or more scaffold structures.

In yet another embodiment of the present invention, one or more scaffold structures are first coated with a polymer solution, for example, a 20% solution of polyurethane-silicon copolymer with 1% fluorine content in the mixed solvent of tetrahydrofuran and N,N-Dimethylacetamide by volume ratio of 75:25; after drying, a graft material comprising the inventive co-extruded filament is placed to contact an outside portion of the coated one or more scaffold structures and then heated to attach the graft material to the outside portion of the coated one or more scaffold structures.

The present invention also provides a reinforced fiber graft material. The reinforced fiber graft material comprises wear-resistant beads and weaves of flexible polymeric fibers. The wear-resistant beads are attached to the flexible polymeric fibers.

The flexible polymeric fiber may be a fiber of any polymer that is flexible and lubricous. Polymers suitable for the flexible polymeric fiber of the inventive reinforced fiber graft include, but are not limited to: polyamide, polyester, polyolefin, and fluorinated polymer. Examples of the polymers suitable for the flexible polymeric material include, but are not limited to: nylon 6, nylon 66, nylon 11, nylon 12, polyethylene terphthalate, polybutylene terephthalate, low density polypropylene, low density polyethylene, and poly(vinylidene fluoride). The terms “low density polypropylene” and “low density polyethylene” are the same as defined hereinabove. Preferably, the flexible polymeric fiber is a fiber of polyethylene terphthalate, or polybutylene terephthalate.

The wear-resistant beads may be polymeric beads. The polymeric beads may be beads of any polymer that is stiff and wear-resistant. Polymers suitable for the wear-resistant polymeric beads of the inventive reinforced fiber graft include, but are not limited to: polyolefin, polyester, poly(ether amide), poly(ether ester), poly(ether urethane), poly(ester urethane), poly(ethylene-styrene/butylene-styrene) and other block copolymers. The terms “ultra high molecular weight polyethylene” and “ultra high molecular weight polypropylene” are the same as defined hereinabove. Preferably, the polymeric beads are beads of ultra high molecular weight polyethylene or ultra high molecular weight polypropylene. The wear-resistant beads may also be ceramic beads.

In the present invention, the flexible polymeric fibers impart flexibility and lubricity to the inventive reinforced fiber graft material, while the wear-resistant beads impart strength and durability to the inventive reinforced fiber graft material without significantly increasing the wall thickness thereof. Thus, the inventive reinforced fiber graft synergistically combines flexibility and durability, and thereby is particularly suitable to be used in vascular grafts or other graft devices. Depending on the intended use or performance requirement, the properties of the inventive reinforced fiber graft material, such as flexibility and durability, may be controlled by components selection and/or number of the wear-resistant beads employed in the inventive reinforced fiber graft. That is, the properties of the inventive reinforced fiber graft may be tuned by using fibers of different flexible polymeric material and beads of different wear-resistant polymeric material, and/or varying the number of the wear-resistant beads in the inventive reinforced fiber graft material.

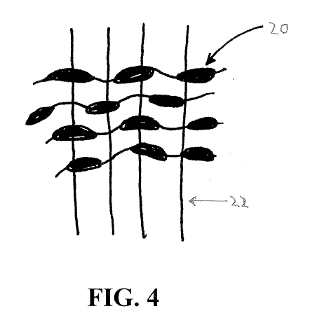

FIG. 4 is a pictorial illustration of a portion of one embodiment of the inventive reinforced fiber graft wherein the wear-resistant beads are attached to the horizontal direction of the weaves of flexible polymeric fibers. In FIG. 4, reference number 20 denotes a wear-resistant bead, and reference number 22 denotes a flexible polymeric fiber.

The inventive reinforced fiber graft material may be prepared by forming weaves of flexible polymeric fibers and then attaching wear-resistant beads to the flexible polymeric fibers, or by attaching wear-resistant beads to flexible polymeric fibers and then forming weaves of the flexible polymeric fibers with the wear-resistant beads attached thereto. Weaves of flexible polymeric fibers with or without wear-resistant beads attached thereto may be formed utilizing any number of techniques described hereinabove or otherwise known in the art, such as weaving, knitting, and braiding. It is understood that details of above described processes are readily ascertainable to one skilled in the art.

Depending on the intended use or performance requirement, the reinforced fiber graft material may in any shape or thickness. It is preferred that the fiber graft material is such a shape that it can be attached to an outside portion of one or more scaffold structures tightly. When the one or more scaffold structures comprise one or more stent segments, it is preferred that the reinforced fiber graft material is in a tubular shape. The wall thickness of the reinforced fiber graft material is determined primarily by weave density and yarn thickness or bulkiness. It is desirable to have the reinforced fiber graft material which is packed tight enough to prevent significant blood seepage, but not so tight that the yarn or fiber bundles pile up on each other. It is preferred that the reinforced fiber graft material has a wall thickness of 0.005 inches or less with a wall thickness of 0.003 inches or less more preferred.

While the present invention has been particularly shown and described with respect to preferred embodiments thereof, it will be understood by those skilled in the art that the foregoing and other changes in forms and details may be made without departing from the spirit and scope of the invention. It is therefore intended that the present invention not be limited to the exact forms and details described and illustrated but fall within the scope of the appended claims.

1-10. (canceled)

11. A co-extruded filament comprising a polymeric inner core and a polymeric outer sheath, wherein the polymeric inner core comprises a flexible polymeric material and the polymeric outer sheath comprises a wear-resistant polymeric material, and the melting point of the polymeric outer sheath is lower than the melting point of the polymeric inner core.

12. The co-extruded filament of claim 11, wherein the ratio of the polymeric inner core to the polymeric outer sheath by weight is about 1:9 to about 9:1.

13. The co-extruded filament of claim 11 has a denier ranging from about 20 to about 1000 with about 20 to about 300 filaments per bundle.

14. The co-extruded filament of claim 11, wherein the flexible polymeric material is a polyamide, a polyester, a polyolefin, or a fluorinated polymer.

15. The co-extruded filament of claim 11, wherein the wear-resistant polymeric material is a polyolefin, a polyester, a poly (ether amide), a poly (ether ester), a poly (ether urethane), a poly (ester urethane), or a poly (styrene-ethylene/butylene-styrene).

16. The co-extruded filament of claim 11, wherein the flexible polymeric material is nylon 6, nylon 66, nylon 11, nylon 12, polyethylene terphthalate, polybutylene terephthalate, low density polypropylene, low density polyethylene, or poly(vinylidene fluoride).

17. The co-extruded filament of claim 11, wherein the wear-resistant polymeric material is ultra high molecular weight polyethylene or ultra high molecular weight polypropylene.

18. The co-extruded filament of claim 11, wherein the polymeric outer sheath further comprises a biodegradable polymer.

19. The co-extruded filament of claim 18, wherein the biodegradable polymer is selected from the group consisting of polyvinyl pyrrolidone, polyethylene glycol, polyethylene oxide, polyvinyl alcohol, polyglycol lactic acid, polylactic acid, polycaprolactone, polydioxanone, and polyamino acid.

20. The co-extruded filament of claim 11, wherein the polymeric outer sheath further comprises one or more biologically active molecules.

21. The co-extruded filament of claim 20, wherein the one or more biologically active molecules are selected from the group consisting of anti-thrombogenic agents, immuno-suppressants, anti-neoplastic agents, anti-inflammatory agents, angiogenesis inhibitors, and protein kinase inhibitors.

22. The co-extruded filament of claim 20, wherein the one or more biologically active molecules are selected from the group consisting of heparin, albumin, streptokinase, tissue plasminogin activator (TPA), urokinase, rapamycin, paclitaxel, and pimecrolimus.

23. A process for assembling a graft device comprising:
providing one or more scaffold structures;
providing a graft material fabricated from a co-extruded filament, the co-extruded filament comprises a polymeric inner core and a polymeric outer sheath, wherein the polymeric inner core comprises a flexible polymeric material and the polymeric outer sheath comprises a wear-resistant polymeric material, and the melting point of the polymeric outer sheath is lower than the melting point of the polymeric inner core;
placing the graft material in contact with an outside portion of the one or more scaffold structures to form a scaffold-graft assembly; and
heating the scaffold-graft assembly to affix the graft material to the outside portion of the one or more scaffold structures.

24. The process of claim 23, wherein each of the one or more scaffold structures comprises one or more stent segments.

25. A reinforced fiber graft comprising wear-resistant beads and weaves of flexible polymeric fibers, wherein the wear-resistant beads are attached to the weaves of flexible polymeric fibers.

26. The reinforced fiber graft of claim 25, wherein the flexible polymeric fiber is a fiber of polyamide, polyester, polyolefin, or fluorinated polymer.

27. The reinforced fiber graft of claim 25, wherein the flexible polymeric fiber is a fiber of nylon 6, nylon 66, nylon 11, nylon 12, polyethylene terphthalate, polybutylene terephthalate, lower molecular weight polypropylene, lower molecular weight polyethylene, or poly(vinylidene fluoride).

28. The reinforced fiber graft of claim 25, wherein the wear-resistant bead is a polymeric bead.

29. The reinforced fiber graft of claim 28, wherein the polymeric bead is a bead of polyolefin, polyester, poly(ether amide), poly(ether ester), poly(ether urethane), poly(ester urethane), or poly(styrene-ethylene/butylene-styrene).

30. The reinforced fiber graft of claim 28, wherein the polymeric bead is a bead of ultra high molecular weight polyethylene or ultra high molecular weight polypropylene.

31. The reinforced fiber graft of claim 25, wherein the wear-resistant bead is a ceramic bead.
